(19) United States
(12) Patent Application Publication
(10) Pub. No.: US 2005/0164405 A1
(43) Pub. Date: Jul. 28, 2005

(54) Non-specific "bridge" link specific "sandwich" immuno-complex to the solid phase in the lateral flow immunoassay

(76) Inventor: Wei Zhao Lu, San Diego (US)

Correspondence Address: Wei Zhao Lu;LuSys Laboratories, Inc., Suite 118, 10054 Mesa Ridge Court, San Diego, CA, 92121, US

(21) Appl. No.: 10/957,892

(22) Filed: Oct. 4, 2004

Related U.S. Application Data
(60) Provisional application No. 60/540,098, filed on Jan. 27, 2004.

(51) Int. Cl. G01N 33/558; G01N 33/543

(52) U.S. Cl. 436/514

(57) ABSTRACT

A chromatographic immunoassay test strip comprising of a solid support having the portions with said portions being in a strip so as to permit capillary flow communication with each other based on the principle of “Bridge-Sanwhich”. In this chromatographic immunoassay test strip herein the analyte in the sample reacts with the ligand to form “ligand A-analyte-ligand B/tracer” first, then is captured by a bridge immobilized on the test zone of solid phase to form a complex of “bridge-ligand A-analyte-ligand B/tracer” which can be detected by vision or equipment.

### REFERENCES CITED

U.S. Patent Documents

| | | |
|---|---|---|
| 4703017 | October, 1987 | Campbell |
| 4,855,240 | Aug. 8, 1989 | Rosenstein; Robert |
| 436/501 5,081,013 | January, 1992 | Rovelli |
| 5,591,645 | January, 1997 | Rosenstein |
| 6,352,862 B1 | Mar. 5, 2002 | Davis |
| 6,485,982 B1 | November, 2002 | Charlton |

### BACKGROUND OF INVENTION

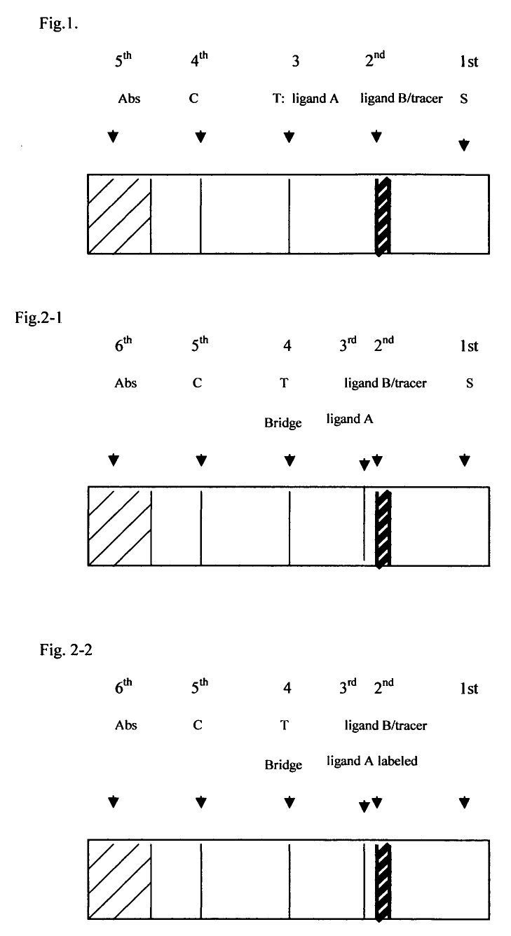

Assays for various analytes have been accomplished by a solid phase assay. Chromatographic immunoassay is as generally known in the art. U.S. Patents (see References cited) discloses a test strip representative of this type (FIG. 1). In such assay, a solid support has five portions and the portions being in capillary flow communication with each other whereby material flows by capillarity. The first and second portions are positioned on the solid support in a manner such that the first portion may be contacted with the material, including any analyte, with material in said first portion being transported by capillarity from the first portion of the support to the second portion thereof and so on. The second portion of the solid support includes a ligand B which is able to react with at least the analyte. The second portion comprised of a ligand portion and a detectable label portion which was conjugated to the ligand B portion to form the ligand B/tracer. The ligand B/tracer portion is reacted with the analyte first after adding the sample. The ligand B/tracer is supported on the solid support in a manner such that when wetted, the ligand B/tracer is capable of being transported by capillarity flow to the other portion of the solid support. Another ligand A to the analyte is immobilized on 3^{rd} portion of said test region. In the case where the lateral flow is in the process, the “ligand B/tracer-analyte” complex will be bound on the test region where a “ligand A-analyte-ligand B/tracer” complex will be formed. Thereafter, depending on the presence and/or absence of analyte and/or the amount of analyte, a T line may be detectable with or without equipment.

A traditional chromatographic immunoassay test strip comprises of a solid support having five portions with said portions being in the same plane so as to permit capillary flow communication with each other based on the principle of “ligand A-analyte-ligand B/tracer” which usually called a “sandwich” immuno-assay, as shown in FIG. 1-1:

1. Sampling adding region; 2. Solid but movable “ligand B/tracer conjugate”; 3. Immobilized ligand A on the Test line of membrane (T line); 4. immobilized non-specific material on the control line of the membrane (C line); 5. Absorbent pad. The solid phase membrane in the device is pre-coated with a specific anti-analyte ligand (call “ligand A”) on the Test zone—3^{rd} region. During the testing, the sample is added to the sample well on 1^{st} region and allowed to flow up through the device by means of capillary flow. The analyte in the sample if it is present reacts with a detectable conjugate of another specific ligand (ligand B/tracer) which was pre-dried on the strip, and an “analyte-ligand B/tracer” will be formed starting in the 2^{nd} region. The mixture then moves along the strip and will be captured by ligand A immobilized on the Test line to form “ligand A˜analyte˜ligand B/tracer complex” on 3^{rd} region. The density of the tracer on T line will be proportional with the concentration of analyte in the samples. The ligand A and ligand B herein is specific to the analyte.

The diagnostics methods based on the principle of testing “antibody-antigen-antibody complex” or said “ligandd A-analyte-ligand B/tracer” usually be named as “Sandwich assay”

### SUMMARY OF THE INVENTION

Invention

The purpose of the present invention is to create a new format of the chromatographic immunoassay. The present invention is to provide an alternative, more sensitive and more specific solid phase assay for determining an analyte. The new format of chromatographic immunoassay assay herein is to form “ligand A-analyte-lingand B/tracer” complex in flow phase by first adding the sample, then “ligand A-analyte-lingand B/tracer’ complex will be captured by a “Bridge” immobilized on T line when the coplex passes pass through the T line.

The invention herein is to immobilize a non-specific material which will react with ligand A, but not react with analyt on the Test line.

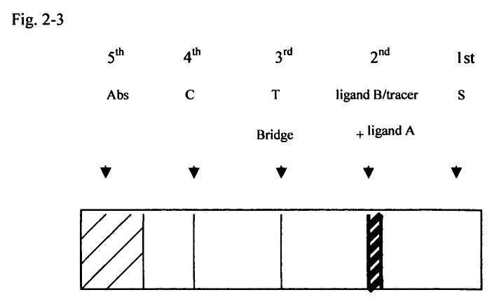

The first type chromatographic immunoassay test strip herein consist of the following as shown in FIG. 2-1:

1. Sampling area; 2. Solid but movable “ligand B/tracer”; 3. Solid but movable ligand A located between the Test line and ligand B/tracer; 4. Immobilized non-specific bridge on the Test line of membrane (T line); 5. immobilized non-specific material on the control line of the membrane (C line); 6. Absorbent pad.

The solid phase-membrane in the device is pre-coated with a non-specific “bridge” but can react with ligand A (call “bridge”) on the Test zone—4^{th} region. The ligand A is in the solid format but is movable when wetted on the membrane in the 3^{rd} region of the device. During the testing, the sample is added to the sample well—1^{st} region and allowed to flow along the device by means of capillary flow. The analyte in the sample if it is present will wet and react with a conjugate of the ligand B/tracer which had been located in the 2 nd region of the strip, and an complex of “analyte˜ligand B/tracer” will be formed. When the “analyte˜ligand B/tracer complex” moves along the strip to ligand A line, a “ligand A˜analyte-ligand B/tracer” will be formed. Then the “ligand A˜analyte-ligand B/tracer” will be captured by the bridge on the Test zone (call “T line”). The density of color on T line will be proportional with the concentration of analyte in the samples. The ligand A and ligand B herein is reacted to the analyte. The bridge however is not reacted to the analyte.

The second type of chromatographic immunoassay test strip herein also has six portions but both ligand A and B are labeled. The strip consist of the following As shown in FIG. 2-2:

1. Sampling area; 2. Solid but movable “ligand B/tracer”; 3. Solid but movable ligand A labeled and located between the Test line and ligand B/tracer; 4. Immobilized non-specific bridge on the Test line of membrane (T line); 5. immobilized non-specific material on the control line of the membrane (C line); 6. Absorbent pad.

The solid phase-membrane in the device is pre-coated with a non-specific “bridge” which can react with ligand A on the Test zone—4^{th} region. The ligand A is in the labeled format but in solid/movable form on the membrane on the 3^{rd} region of the device. During the testing, the sample is added to the sample well—1 ^{st} region and allowed to flow along the device by a capillary flow. The analyte in the sample if it is present will wet and react with a conjugate of a ligand B/tracer, which is located in the 2nd region of the strip, and an “analyte˜ligand B/tracer” will be formed. When the “analyte˜ligand B/tracer complex” moves along the strip to the ligand A line, a “ligand A/labeled˜analyte-ligand B/tracer” will be formed. Then the “ligand A/labeled˜analyte-ligand B/tracer” will be captured by the bridge on the Test zone (call “T line”). The density of color on T line will be proportional with the concentration of analyte in the samples. The ligand A and lignad B herein is reacted to the analyte. But the bridge is not reacted to the analyte. In this case, the different color will be shown up at T line depended which color label be applied to ligand while the analyte is present or absent in the sample.

The Third type of chromatographic immunoassay test strip herein has five portions as FIG. 2-3 shows.

1. Sampling area; 2. Solid but movable “ligand A+ligand B/tracer; 3. Immobilized non-specific bridge on the Test line of membrane (T line); 4. immobilized non-specific material on the control line of the membrane (C line); 5. Absorbent pad.

The solid phase-membrane in the device is pre-coated with a non-specific “bridge” which can react with ligand A on the Test zone—4^{th} region. The ligand A (labeled or unlabeled) was mixed with ligand B/tracer in the device. During the testing, the sample is added to the sample well—1^{st} region and allowed to along the device by means of capillary flow. The analyte in the sample if it is present will form a conjugate of either “ligand A-analyte” or “ligand B/tracer-analyte” or “ligand A-analyte-ligand B/tracer”. When the mixture of conjugate moves upward to the bridge—3^{rd} line the “ligand A˜analyte-ligand B/tracer” will be captured by the bridge on the Test zone (call “T line”). The density of color on T line will be proportional with the concentration of analyte in the samples. The ligand A and ligand B herein is reacted to the analyte. But the bridge is not reactive to the analyte at all.

The diagnostics methods based on the principle of testing “antibody-antigen-antibody complex” or said “ligand A-analyte-ligand B labeled” is usually called a “Sandwich assay”. The invention herein is named as a “Bridge-Sandwich assay”.

In accordance with the present invention, there is provided a solid support having at least a the portion for adding the sample, and a second portion for ligand B/tracer, a third portion for ligand A, a fourth portion for the binder which might capture “ligand A-analyte-lingand B/tracer complex” to form a detectable line, a fifth portion for control, and a sixth portion for absorbing liquid traveling through the porous materials from the liquid flow.

The solid support employed in the assay which provides the capillary flow paths for ligand A, ligand B/tracer and analyte are on one strip. The solid support also provides a surface area capable of supporting the binder. As examples, of such materials, there may be but not limited to: glass fiber, cellulose, nylon, various chromatographic paper, nitrocellulose, etc. The solid support is preferably in the sheet form, generally being in the form of a card, a strip or dipstick, etc.

The ligand B which is labeled for use as the tracer in the assay is dependent upon the analyte to be assayed. The ligand B/tracer would be reacted to the analyte in the specimen when it is wet. To produce the tracer, the ligand B may be labeled with a detectable marker. The particular label may be visible includes but is not limited to a dye or any colored substance, such as gold particle, colored latex, liposome, erythrocytes, polymer particles, bacterial and other materials. The label may be non-solid labels, such as radiotopes, enzymes, fluorescent compounds or other chromogen labels, dyes or chemiluminescent materials that either produce or catalyze a color-developing reaction which may be detected with or without further treatment and with or without the use of instrumentation.

The strip device provide the flow through of analyte and ligand A and ligand B/tracer by capillary attraction from the first portion to the other portion of the solid support. In addition, the solid support is one which is capable of supporting the ligand A, ligand B/tracer and other binders. Porous capillarity-possessing materials are suitable for use as solid support, such as glass fiber, nylon, chromatographic papers, nitrocellulose, etc.

The ligand A is a ligand to analyte and must be a hetero ligand comparing with ligand B, such as ligand B is mouse monoclonal antibody specific to hCG, ligand A must use goat or rabbit anti-hCG antibody. The ligand A is solidized and located close to ligand B/tracer and is movable along with the liquid flow. The ligand A will be formed “ligand A-analyte-ligand B” complex when meeting the “analyte-ligand B”. The bridge on the Test line is supported on a solid support in an appropriate concentration, as herein above described, can capture the sandwich of “ligand A-analyte-ligand B/tracer” or “ligand A-analyte”. The activity of bridge is not related to the analyte and ligand B, but must have a reaction activity with the ligand A. For example, the bridge is an antibody to ligand A. The ligand B/tracer is antibody to the analyte labeled with a detectable label, such as a gold particles. The amount of ligand B/tracer which is bound to the bridge on the solid support through the ligand A is directly proportional to the amount of analyte in the sample, and the presence and/or amount of analyte present in the sample may be determined from the presence and/or amount of tracer which becomes bound to the support through the analyte.

The present invention is applicable to detecting a wide variety of antigen analytes, such as: hCG, LH, FSH, TSH, PSA, CEA, a-FP, Troponin I, CKMB, Myoglobin, infectious antigens etc. The present invention is applicable to detecting a wide variety of antibody analyte, such as antibodies to infectious antigens and auto-antibodies.

### EXAMPLE 1

hCG Test According to FIG. 2-1

The hCG chromatographic immunoassay test strip herein has six portions. The solid phase-membrane in the device is pre-coated with a non-specific antibody but can react with ligand A, such as donkey anti-goat antibody as a capture antibody (bridge) on the Test zone—4^{th} region. The ligand A, such as goat anti-hCG, was pre-coated on the solid but in movable form on 3^{rd} region in the device. During the testing, the sample is added to the sample well—1^{st} region and allowed to flow up through the device by a capillary flow. The sample will wet the conjugate and the hCG antigen if present in the sample react with a colored gold conjugate of a specific mouse anti-hCG antibody on the 2 nd region of the strip, and an antigen-antibody complex—“antigen hCG˜mouse monoclonal anti-β-hCG antibody-gold” will be formed. Then the liquid flow will wet and move toward to the ligand A and form “goat anti-hCG antibody˜hCG˜mouse monoclonal anti-β-hCG antibody-gold” and then captured by the bridge to form “donkey anti-goat antibody˜goat anti-hCG antibody˜hCG˜mouse monoclonal anti-β-hCG antibody-gold” on the T line—region 4th of the membrane. The color of T line will be seen by vision if hCG antigen if present in the sample. The density of color on T line will be proportional with the concentration of hCG in the samples

Materials Needed

1) 10 mil Matte vinyl board with adhesive on one side (G & L, San Jose, Calif.)
2) nitrocellulose membranes (Saitorius, Edgewood, N.Y.)
3) bibulous paper (Whatman, Fairfield, N.J.)
4) glass fiber (Pall-Gelman, Ann Arbor, Mich.)
5) gold chloride (Sigma, St. Louis, Mo.)
6) hCG antigen (Sigma, St. Louis, Mo.)
7) mouse anti-hCG (Biopacific, Emeryville, Calif.)
8) goat anti-hCG (Maxmed, San Diego, Calif.)
9) donkey anti-goat antibody (Jackson, West Grove, PE)
   Preparation of the Chromatographic Immunoassay Test Strip
1) using reagent dispenser to dispense 4 mg/ml of donkey anti-goat IgG on 25 mm*300 mm nitrocellulose membranes to make test binder (T line);
2) using reagent dispenser to dispense 2 mg/ml goat anti-mouse IgG antibodies on 25 mm*300 mm nitrocellulose membranes to make comparison binder (C Line);
3) using reagent dispenser to dispense 3 mg/ml goat anti-hCG IgG antibodies on 25 mm*300 mm nitrocellulose membranes to make a ligand A line;
4) marking 10 ml gold conjugate with 25 μg mouse anti-β-hCG antibodies;
5) using centrifuge at 12,000 rpm speed to separate the gold conjugate—ligand B labeled/tracer;
6) immersing glass fiber in liquid of gold conjugate, which is dissolved by a kind of phosphate buffer, dry by a vacuum and cut it into 6*30 mm strips;
   Assembling the Test Kit
1) affixing the glass fiber; gold conjugate; nitrocellulose membrane with an a T line, a Control line and a ligand A line on the solid support in a juxtapositioned relationship as a test strip;
2) affixing the aforementioned the test strips into a plastic cassette.

B Testing: add 2 drops of sample into 1^{st} region of FIG. 2 and read the results at 5 min. as shown in the table 1.

TABLE 1
The test results of hCG kit made according to FIG. 2-1

| | HCG | | LH(mIU/ | | FSH(mIU/ | TSH(uIU/ |
|---|---|---|---|---|---|---|
| | (mIU/ml) | | ml) | | ml) | ml) |
| | 0 | 25 | 100 | 300 | 1,000 | 1,000 |
| Shown time of | — | 4 min. | 2 min. | — | — | — |
| Test line | | | | | | |

### EXAMPLE 2

hCG Test According to FIG. 2-2

Use the same materials and procedure as same as Example 1 except the goat anti-hCG are labeled by the latex in advance.

Material used: Latex: white 0.75 micron, 2.53% solids (Polysciences, Inc.)

Preparation of antibody labeled with latex:
1) 1 ml of latex solution wash twice with 10 mM PBS;
2) Add 2 ml of 0.2 mg/ml goat anti-hCG antibody into latex and incubate at 37° C. for 2 hrs;
3) After blocking with 10% BSA and wash twice, soak the latex solution into 5 mm glass fiber, then vacuum dry.

Other procedure are as the same as Example 1. Put the latex strip on the position 3 ccording FIG. 2-2.

The test results is shown in Table 2.

TABLE 2
The test results of hCG kit made according to FIG. 2—2

| HCG mIU/ml | 0 | 50 | 100 |
|---|---|---|---|
| Shown time of | — | 10 min. | 8 min |
| T line | | | |

### EXAMPLE 3

hCG Test According to FIG. 2-3

Use the same materials and procedure as same as Example 1 except mixing the goat anti-hCG antibody with the mouse monoclonal anti-β-hCG antibody gold labeled (add 0.1 mg goat anti-hCG antibody into 1 ml anti-β-hCG antibody gold labeled liquid, then transfer 6 mm×300 mm glass fiber and vacuum dry.

Other procedure are as the same as Example 1 except without region 3.

The test results is shown in Table 3.

TABLE 3
The test results of hCG kit made according to FIG. 2-3

| HCG mIU/ml | 0 | 50 | 100 |
|---|---|---|---|
| Shown time of | — | 10 min. | 5 min |
| T line | | | |

1. A test device for determining an ingredient (herein named as analyte) of a liquid sample, the device comprising a matrix which permitting liquid travel through the solid phase and communication with each other, said matrix containing at least:
(A) 1^{st} region: a sample adding region; and
(B) 2^{nd} region: a solid and movable phase of ligand B/tracer; ligand B is a binder which binds to the analyte. and
(C) 3^{rd} region: a movable ligand A having the location close to or mixed with the ligand B/tracer region; ligand A is also binder to the analyte; ligand A is pre-loaded on the solid support in a solid format and movable; and
(D) 4^{th} region: a test region (call T line) having a “bridge” immobilized on which a “bridge-ligand A-analytes-ligand B/tracer complex” will be formed and detectable.

2. A method of assay for determining the presence of an analyte in a liquid sample, comprising of:
(A) adding a liquid sample to 1^{st} region of the device in claim 1: a sample adding region; and
(B) The analytes if presents in the sample will wet and react with the lingand B/tracer within 2^{nd} region and flow up by capillary flow.
(C) When it meets the movable ligand A within 3^{rd} region, the “ligand A-analyte-lingand B/tracer’ will be formed; and
(D) When the liquid moving toward the test region or 4^{th} region on which the non-specific binder (name “bridge”) was immobilized. In the test region the “bridge-ligand A-analyte-ligand B/tracer complex” will be formed and;
(E) The presence of analyte in the sample will be determinate on 4^{th} region by detecting the presence of “bridge-ligand A-analyte-ligand B/tracer complex” through the vision or by equipment.

3. According claim 1 and 2, the “bridge” is materials immobilized on the Test line. The “bridge” can react with ligand A, but not react with the analyte and ligand B;

4. According claim 1 and 2, the ligand A
(A) is able to bind or react with the analyte;
(B) is either unlabeled or labeled with a visible particles or other detectable materials;
(C) is pre-load on the solid support in the solid format when drying and movable when wetted;
(D) located in the 3^{rd} region or mixed together with ligand B/tracer located in the 2^{nd} region

5. According claim 1 and 2, an application of said test strip for detection of hormones, proteins, other antigens or antibodies but not limited to this application since there are other applications using this procedure for detection of analyte but work along the same principle as said application.
